(19) United States
(12) Patent Application Publication
(10) Pub. No.: US 2011/0002918 A1
(43) Pub. Date: Jan. 6, 2011

(54) METHODS OF TREATING DISEASED TISSUE

(75) Inventor: Jeffrey I. Levatter, Solana Beach, CA (US)

Correspondence Address: KNOBBE MARTENS OLSON & BEAR LLP, 2040 MAIN STREET, FOURTEENTH FLOOR, IRVINE, CA, 92614, US

(73) Assignee: PHOTOMEDEX, Montgomeryville, PA (US)

(21) Appl. No.: 12/718,830

(22) Filed: Mar. 5, 2010

Related U.S. Application Data
(60) Provisional application No. 61/158,079, filed on Mar. 6, 2009.

(51) Int. Cl. A61K 39/395; A61K 31/56; A61K 31/4436; A61K 31/37; A61K 31/122; A61K 31/192; A61K 31/519; A61K 38/13; A61N 5/06

(52) U.S. Cl. 424/133.1; 424/178.1; 424/142.1; 514/169; 514/337; 514/455; 514/680; 514/570; 514/249; 514/18.7; 607/88

(57) ABSTRACT

Treatment protocols for severe psoriasis include administering biologics, stopping all administration of the biologics after the severity of the psoriasis has reduced and has reached an equilibrium, mildness and/or a tolerable state of remission, and administering UV phototherapy. The biologics may include, for example, the biologics found in Amevive®, Enbrel®, Humira®, Raptiva®, and Remicade® and/or alefacept, etanercept, adalimumab, efalizumab, infliximab, and ustekinumab. The UV phototherapy may be repeated, for example daily, weekly, monthly, yearly, to keep the psoriasis in a mild state or in a tolerable state of substantial remission. Parameters for administering UV phototherapy may be determined based on skin tone, Fitzpatrick skin phenotype, the severity of the psoriasis, the area of exposure, and/or the MED.

### RELATED APPLICATIONS

This application claims priority benefit under 35 U.S.C. §119(e) of U.S. Provisional Application Ser. No. 61/158,079, filed Mar. 6, 2009, which is hereby incorporated by reference in its entirety.

### BACKGROUND

1. Field

The present application generally relates to methods of treating skin disorders, and, more specifically, to treatment protocols for psoriasis.

2. Description of the Related Art

Skin disorders, including atopic dermatitis, dyshidrosis, eczema, lichen planus, psoriasis, and vitiligo, are conditions that commonly affect large populations at some time in their lives. For example, about 2% to about 3% of the population of northern Europe is estimated to be afflicted with psoriasis. Although the disease's prevalence in the United States is not as well understood, it appears that between 150,000 and 260,000 new cases are diagnosed each year. This suggests that at least several million people suffer from the disease in the United States.

Psoriasis can range in severity from relatively mild, with some drying and flaking of the affected skin, to severe cases with very severe outbreaks over large areas of the patient's body. Even very mild psoriasis is uncomfortable and unsightly. Severe cases can be physically and psychologically debilitating, presenting a very serious threat to the patient's overall health.

Psoriasis is generally considered a chronic disease condition, and therefore the treatment of this disease is usually targeted for the long term. There are several available treatments each of which has certain advantages and disadvantages. Conventional treatment for psoriasis can generally be divided into two main types: medication and light therapy.

Injectable biologics are a popular method of treating severe psoriasis. Examples of injectable biologics include Amevive®, Enbrel®, Humira®, Raptiva®, and Remicade®. Despite their popularity, however, these biologics can have severe side effects (e.g., nausea, fatigue, difficulty sleeping, vomiting, headaches, easy bruising and bleeding, fever, diarrhea, chills, liver, kidney, and bone marrow damage, and cancer) and, once started, treatment must be continued for the life of the patient. Discontinuing biologic treatment or even reducing the doses of the medication causes the psoriasis to revert to its pre-treatment state. Thus, once a patient begins to receive biologics, administration continues for the rest of his or her life. In some cases, the quantity and/or severity of the psoriasis lesions may become even more severe upon discontinuance or reduction of biologic treatment, which is called a psoriasis “rebound.” Accordingly, once biologic treatment starts, it is never discontinued. Unfortunately, the side-effects of biologic treatment become more severe or likely with prolonged use. In addition, biologics can be very expensive, for example about $20,000 per year.

Light therapy (or phototherapy), which involves exposing the skin to ultraviolet (UV) light, is used to treat skin conditions including psoriasis. In general, phototherapy is considered an effective treatment that permits both rapid control and long-term maintenance. In addition, phototherapy treatments can have long durations (e.g., on the order of tens of minutes or hours) as the UV light is directed at a target, applied, moved, and repeated until the entire affected area has been treated, particularly for large treatment areas. Side effects may include freckling, aging, darkening (i.e., erythema or sunburn), bruising and skin cancer.

### SUMMARY

In certain embodiments, a method of treating a subject having psoriasis comprises administering UV phototherapy to the subject after discontinuing administering a biologic to the subject after improving severity of the psoriasis. In some embodiments, the biologic comprises at least one selected from the group consisting of the biologic found in Amevive®, Enbrel®, Humira®, Raptiva®, and Remicade®. In some embodiments, the biologic comprises at least one selected from the group consisting of alefacept, etanercept, adalimumab, efalizumab, infliximab, and ustekinumab. In some embodiments, the method further comprises repeating administering the UV phototherapy at least once per day, at least once per week, at least once per month, or at least once per year. In some embodiments, parameters for administering the UV phototherapy are at least partially determined based on the skin tone of the subject, Fitzpatrick skin phenotype of the subject, the severity of the psoriasis, the area of exposure, the MED of the subject, or combinations thereof. In some embodiments, the method further comprises continuing administering UV phototherapy to the subject after psoriasis becomes mild or in a tolerable state of remission.

In certain embodiments, a method of treating a subject having psoriasis comprises administering a biologic to the subject, discontinuing administering the biologic to the subject after improving severity of the psoriasis, and after discontinuing, administering UV phototherapy to the subject. In some embodiments, the biologic comprises at least one selected from the group consisting of the biologic found in Amevive®, Enbrel®, Humira®, Raptiva®, and Remicade®. In some embodiments, the biologic comprises at least one selected from the group consisting of alefacept, etanercept, adalimumab, efalizumab, infliximab, and ustekinumab. In some embodiments, the method further comprises repeating administering the UV phototherapy at least once per day, at least once per week, at least once per month, or at least once per year. In some embodiments, parameters for administering the UV phototherapy are at least partially determined based on the skin tone of the subject, Fitzpatrick skin phenotype of the subject, the severity of the psoriasis, the area of exposure, the MED of the subject, or combinations thereof. In some embodiments, the method further comprises continuing administering UV phototherapy to the subject after psoriasis becomes mild or in a tolerable state of remission.

In certain embodiments, a method of treating a subject having psoriasis comprises administering UV phototherapy to the subject after discontinuing administering a biologic to the subject after improving the severity of the psoriasis by at least a predetermined amount. In some embodiments, the predetermined amount is at least about 25%, at least about 40%, at least about 50%, or at least about 70%.

In certain embodiments, a method of treating psoriasis comprises administering identifying a patient on a biologic regimen treating psoriasis, after the biologic regiment reduces severity of the psoriasis, administering a first UV phototherapy to the subject to further treat the psoriasis to be mild or less, and administering a second UV phototherapy to the subject to maintain the psoriasis to be mild or less.

In certain embodiments, a method of treating a subject having severe psoriasis comprises administering a first biologic to the subject, after improving the severe psoriasis, administering a second biologic to the subject, discontinuing administering the second biologic after further improving the severe psoriasis, and after discontinuing administering the second biologic, instructing the subject to undergo UV phototherapy. In some embodiments, the first biologic is the same as the second biologic. In some embodiments, the first biologic is different than the second biologic. In some embodiments, the method further comprises discontinuing administering the first biologic after further improving the severe psoriasis.

In certain embodiments, a method of treating a subject having severe psoriasis comprises administering a first biologic to the subject, discontinuing administering the first biologic to the subject after improving the severe psoriasis, after discontinuing administering the first biologic, administering a first UV phototherapy, administering a second biologic to the subject if the subject relapses or rebounds to severe psoriasis, discontinuing administering the second biologic to the subject after improving the severe psoriasis, and after discontinuing administering the second biologic, administering a second UV phototherapy. In some embodiments, the first biologic is the same as the second biologic. In some embodiments, the first biologic is different than the second biologic.

In certain embodiments, a method of treating psoriasis comprises administering phototherapy to a patient who has stopped taking a biologic that reduced severity of the psoriasis. In some embodiments, the biologic reduced the severity of the psoriasis from severe to moderate. In some embodiments, the biologic reduced the severity of the psoriasis from severe to mild.

In certain embodiments, a method of treating a subject having severe psoriasis comprises administering UV phototherapy to the subject who was previously on a biologic that reduced severity of the psoriasis from severe to moderate or mild. The biologic comprises at least one ingredient selected from the group consisting of alefacept, etanercept, adalimumab, efalizumab, infliximab, and ustekinumab.

In certain embodiments, a method of treating a subject having severe psoriasis comprises administering UV phototherapy to the subject after administering a biologic to the subject to reduce the severity of the psoriasis from severe to moderate or mild, the biologic comprising at least one selected from the group consisting of the biologic found in Amevive®, Enbrel®, Humira®, Raptiva®, and Remicade®.

In certain embodiments, a method of treating a subject comprises identifying an area of diseased tissue, administering a biologic to the subject to treat the diseased tissue; discontinuing administering the biologic to the subject after reducing the area of diseased tissue, and after discontinuing administering the biologic, administering UV phototherapy to the subject to further treat the diseased tissue. In some embodiments, discontinuing administering the biologic is after the area is reduced from severe to moderate. In some embodiments, discontinuing the biologic is after the area is reduced from severe to mild. In some embodiments, discontinuing the biologic is after the area decreases at least about 25%, at least about 40%, at least about 50%, or at least about 70%. In some embodiments, discontinuing administering the biologic is after the area is less than about 10%, less than about 5%, or less than about 3% of the total body epidermal area of the subject. In some embodiments, discontinuing administering the biologic is after the area is less than about 2,000 cm², less than about 1,000 cm², or less than about 500 cm².

In some embodiments, a method of treating psoriasis comprises identifying a patient on a biologic regimen treating psoriasis and after the biologic regiment reduces the severity of the psoriasis, administering UV phototherapy to the subject. In some embodiments, the method further comprises, after the biologic reduces severity of the psoriasis, instructing the patient to discontinue the biologic regimen.

For purposes of summarizing the invention and the advantages achieved over the prior art, certain objects and advantages of the invention are described herein. Of course, it is to be understood that not necessarily all such objects or advantages need to be achieved in accordance with any particular embodiment. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the present disclosure are described with reference to the drawings of certain embodiments, which are intended to illustrate certain embodiments and not to limit the invention.

FIGS. 1A to 1C display example embodiments of diseased tissue treatment protocols.

FIGS. 2A to 2C display further example embodiments of diseased tissue treatment protocols.

FIGS. 3A to 3C display further example embodiments of diseased tissue treatment protocols.

### DETAILED DESCRIPTION

Although certain embodiments and examples are described below, those of skill in the art will appreciate that the invention extends beyond the specifically disclosed embodiments and/or uses and obvious modifications and equivalents thereof. Thus, it is intended that the scope of the invention herein disclosed should not be limited by any particular embodiments described below.

The present disclosure generally relates to methods of treating a subject having psoriasis. However, the methods described herein may equally apply to other diseases (e.g., atopic dermatitis, dyshidrosis, eczema, lichen planus, psoriasis, vitiligo) that are treated with biologics on a long-term basis now or in the future and/or to non-human subjects. In some preferred embodiments, severe psoriasis is reduced to a state of tolerable remission or equilibrium. Such methods may comprise administering a biologic to the subject, discontinuing administering the biologic to the subject after improving the severe psoriasis, and after discontinuing, administering UV phototherapy to the subject in a maintenance mode over the long term. UV phototherapy may also be used after discontinuing the biologics to improve the percentage of cleared body surface area in order to allow the light device to maintain stability and equilibrium of the patient at an improved level of clearing.

Psoriasis is a chronic condition affecting various tissues including the skin and joints. It occurs, for example, on the skin of the scalp, face, elbows, knees, back, abdomen, knee, hand, foot, and the nails. Psoriasis may also occur on joints of knees, spine, and sacroiliac to cause psoriatic arthritis. The present disclosure is generally related to a treatment of any tissue that has psoriasis symptoms. Although generally described herein as treating the area of diseased tissue, the normal tissue, for example proximate to the diseased tissue, can also be treated. In some embodiments in which the disease is widespread, the treatment may be extended to substantially the entire surface area of the body.

Psoriasis can be divided into various types according to the affected area and/or symptoms. For example, plaque psoriasis (i.e., psoriasis vulgaris) is a common form of the condition and accounts for about 80% to about 90% of patients. Plaque psoriasis typically appears as red patches or plaques with dry, silvery scales. Another type is guttate psoriasis, which is characterized by numerous small round spots. Guttate psoriasis often renders these numerous round spots in large areas of the body, such as the trunk, limbs, and scalp. Flexural psoriasis (i.e., inverse psoriasis), on the other hand, appears as smooth inflamed patches of skin. It occurs in skin folds such as areas around the genitals, the armpits, the overweight stomach, and the breasts. Pustular psoriasis appears as raised bumps and is commonly found locally in the hands and feet, but it can extend to other parts of the body. Erythrodermic psoriasis usually comes with severe itching, swelling, and pain. These radical symptoms may involve the widespread inflammation and exfoliation of the skin. Fingernails and toenails may be affected by nail psoriasis, and often undergo a variety of changes in the appearance. Small indents in the nails (i.e., pitting), lifting up of the nails, discoloration, thickening, and crumbling of nails may appear due to nail psoriasis. In addition to skin, psoriasis can affect joints and some connective tissues to cause psoriatic arthritis. Psoriatic arthritis can affect any joint of the fingers, toes, hips, knees, and spine and cause swelling and pain. Embodiments disclosed herein may be used to treat any type or combination of types of psoriasis, some of which are described above. In certain embodiments, the methods described herein may be used to treat one specific type of psoriasis. In certain alternative embodiments, the methods described herein may be used to treat two or more types of psoriasis.

The severity of psoriasis can be classified or “scored” in a variety of ways. This disease varies from causing relatively minor plaques in a localized area of the body to covering substantially large area of the body. In a classification method that is based on the surface area of tissue affected, psoriasis can be graded as mild (e.g., affecting less than about 3% of the total area of the body surface), moderate (e.g., affecting about 3% to about 10% of the total area of the body surface), or severe (e.g., affecting more than about 10% of the total area of the body surface). Other scales may also be employed for measuring the severity of psoriasis. For example, in addition to the size of affected or influenced body surface area, factors such as the condition period, the frequency of disease recurrence, disease activity (e.g., degree of plaque redness, thickness, and scaling), response to previous therapies, and the impact of the disease on the person may also be considered to determine the severity of the disease. Therefore, a subject having less than 3% of the total body surface area affected by the condition may be considered to have moderate or severe psoriasis if the affected area accompanies radical symptoms such as swelling or pain. Alternatively, a subject having psoriasis condition that is resistant or recalcitrant to one or more known treatments may also be considered to have severe psoriasis regardless of the size of influenced area. Therefore, psoriasis may be characterized as severe if at least one of the following is observed: the area of influenced tissue is greater than about 10% of the total body surface area; the condition continues as long as a month or even a year; the disease activity is substantially active; and the disease is resistant to one or more of known treatments. Psoriasis may also be considered severe if the diseased area comprises between about 10% and about 20% of the total body surface area of the subject, between about 20% and about 30% of the total body surface area of the subject, or greater than about 30% of the total body surface area of the subject. Certain embodiments disclosed herein may be used to treat psoriasis of various degrees of severity. Some embodiments may be used to treat mild, moderate, or severe psoriasis. Some embodiments may be used to treat any combination of mild, moderate, and severe psoriasis. Some embodiments may be used to treat mild to moderate psoriasis and/or moderate to severe psoriasis.

It has been generally established that psoriasis may result from a disorder in the immune system. More particularly, research indicates that psoriasis is a T-cell mediated disease and that the skin hyperplasia of the disease is a result of abnormal immune system activation. The use of biologics, including injectable biologics to treat psoriasis is well known. Examples of such injectable biologics include the biologics found in Amevive®, Enbrel®, Humira®, Raptiva®, and Remicade®. Biologics are generally produced from a cell or living organisms and/or based on human or animal proteins, but may also include some synthetic materials. In addition, active compositions of the foregoing example biologics such as alefacept, etanercept, adalimumab, efalizumab, infliximab, and ustekinumab may also be included as the injectable biologics used herein. In some embodiments, the biologics may be anti-tumor necrosis factor (anti-TNF) drugs. Other biologics, including injectable biologics, that are effective to treat psoriasis, whether now known or developed in the future, may also be used. The examples listed above may also be combined with or substituted with/replaced by one or more of, steroids, vitamins, coal tar, retinoid, calcipotriene, tazarotene, psoralens, anthralin, acitretin, methotrexate, cyclosporine, non-biologic pharmaceutical drugs, and the like, whether now known or developed in the future.

Injectable biologics are known to cause undesirable side effects, including hormonal imbalance, internal organ damage, elevated blood pressure, allergic reactions, and cancer. Therefore, the biologics would ideally be limited to minimal doses and short-term treatment. However, such restrictive use of the biologics is heretofore thought impossible because the reduction in doses or discontinuation of medication is known to cause recrudescence of the psoriasis condition, even to a more severe degree than prior to the treatment. Thus, administration of the biologics continues for the rest of the life of the patient once it begins, despite the side effects and the increased risk thereof due to the long-term application. Thus, while administration of the biologic is used to decrease the severity of the disease (e.g., from severe to moderate, from severe to mild, from 30% coverage to 15% coverage), it is a poor long-term solution due to the grave, possibly lethal side effects.

The biologics may be administered to the subject via parenteral administration. For parenteral applications, the biologics may be injected under the skin, into the muscle, and into the vein or lymph vessels. Certain biologics designed for oral and/or topological administration may also be designed for parenteral administration, and vice versa.

A dosage of the biologics and/or a frequency of its application can generally be determined upon numerous factors including the body weight of the subject, the age, the sex, the general health condition of the subject, the severity of the disease, the area of diseased epidermal tissue, the likelihood and/or severity of side effects, the sensitivity of the subject to the biologics, the desired biologics administration duration, the medical expenses, any specific genetic, physiological, or medical condition which may not be compatible to certain biologic treatments (e.g., pregnancy), or any combinations thereof. The biologics can be administered to the subject at least once per day, once per week, once per month, once per year or any combinations thereof. In some embodiments, the dosage and/or frequency are modified over the course of administration. Such modifications may depend on factors including, but are not limited to, the realized effect of the biologics on the disease condition, the general health of the subject, the development and severity of side effects, and the compatibility of the treatment with the subject's environment. In certain embodiments, the dosage and/or frequency may be increased, for example to enhance the treatment effect on the disease over the course of treatments. In certain embodiments, the dosage and/or frequency may be decreased over the course of treatments, for example when the disease condition improves more than expected. In certain embodiments, the dosage and/or frequency may be increased during the early stage of treatment and decreased during the later stage. In certain embodiments, the dosage may be increased over the course of treatments while the frequency may be decreased over the course of treatments. In certain other embodiments, the dosage may be decreased over the course of treatments while the frequency may be increased over the course of treatments.

In certain embodiments, administering biologics comprises administering two or more biologics to the subject. When two or more biologics are administered to the subject, the biologics can be administered in parallel, in series, or a combination thereof. In certain embodiments, a first biologic is administered until psoriasis is reduced from severe to moderate, administration of the first biologic is discontinued, a second biologic is administered until the psoriasis is reduced from moderate to mild, and administration of the second biologic is discontinued.

In compliance with certain protocols described herein, the biologics are administered for a certain period, and are then discontinued. In some embodiments, the limited duration of administration of the biologic is based at least in part on a time period, for example one or more hours, one or more days, one or more weeks, one or more months, or one or more years. In some embodiments, the limited duration of administration of the biologic is based at least in part on the realized effect of biologics. In certain such embodiments, administration of the biologics may be discontinued after severe psoriasis becomes moderate or mild. In certain such embodiments, administration of the biologics may be discontinued after moderate psoriasis becomes mild.

In some embodiments, administration of the biologics may be discontinued after the severity of the disease can be maintained by continued use of the biologics. In some embodiments, the biologics may be used like triage to make the disease condition under the control of treatment, and the other protocols such as UV phototherapy may follow to improve the disease condition. In some embodiments in which the psoriasis accompanies radical symptoms such as swelling, pain, and/or itching, the biologics may be administered at least until such symptoms become stable or reduced. In some other embodiments, the biologic may be discontinued once the patient stops feeling pain or itching in the diseased area even if surface area of diseased tissue remains substantially same. In some of such embodiments, the biologics may be administered to stop the spread of the psoriasis to normal tissues, and then becomes discontinued. In some embodiments in which the patient suffers from the psoriasis that is relatively quickly spreading from one tissue to another, the biologics may be administered until the disease stops spreading or at least spreads slowly. Once this spreading stops or slows down, the biologics treatment may be discontinued.

In some embodiments, administration of the biologics may be discontinued after the area of psoriasis decreases by a certain percentage, for example at least about 5%, 10%, 15%, 25%, 30%, 40%, 50%, 60%, 70%, 80%, 90%, 95%, or values therebetween. In some embodiments, administration of the biologics may be discontinued after a decrease in severity of the psoriasis from covering more than about 30% of the total body epidermal area of the subject to less than about 30% of the total body epidermal area of the subject, from covering more than about 25% of the total body epidermal area of the subject to less than about 25% of the total body epideimal area of the subject, from covering more than about 20% of the total body epidermal area of the subject to less than about 20% of the total body epidermal area of the subject, from covering more than about 15% of the total body epidermal area of the subject to less than about 15% of the total body epidermal area of the subject, from covering more than about 10% of the total body epidermal area of the subject to less than about 10% of the total body epidemal area of the subject, from covering more than about 9% of the total body epidermal area of the subject to less than about 9% of the total body epidermal area of the subject, from covering more than about 8% of the total body epidermal area of the subject to less than about 8% of the total body epidermal area of the subject, from covering more than about 7% of the total body epidermal area of the subject to less than about 7% of the total body epidermal area of the subject, from covering more than about 6% of the total body epidermal area of the subject to less than about 6% of the total body epidermal area of the subject, from covering more than about 5% of the total body epidermal area of the subject to less than about 5% of the total body epidermal area of the subject, from covering more than about 4% of the total body epidermal area of the subject to less than about 4% of the total body epidermal area of the subject, from covering more than about 3% of the total body epidemal area of the subject to less than about 3% of the total body epidermal area of the subject, from covering more than about 2% of the total body epidermal area of the subject to less than about 2% of the total body epidermal area of the subject, from covering more than about 1% of the total body epidermal area of the subject to less than about 1% of the total body epidermal area of the subject, and combinations thereof (e.g., from covering more than about 30% of the total body epidermal area of the subject to less than about 10% of the total body epidermal area of the subject).

Certain protocols described herein further comprise administering UV phototherapy. UV phototherapy is generally administered after discontinuing the administration of the biologics in some embodiments. In some other embodiments, however, UV phototherapy may be administered before discontinuing the administration of the biologics (e.g., to treat problematic lesions, as a supplement to the biologics). Once the supplemental purpose is achieved, the biologic is discontinued, and UV phototherapy is applied to maintain equilibrium.

UV phototherapy, which involves exposure of the skin to UV light, has also been considered an effective treatment method of psoriasis. Phototherapy, may improve the disease condition in somewhat different ways from the biologics. For example, certain biologics may suppress the immune system, in particular the activity of T-cells. While certain UV phototherapy may also be able to modify the immune system by regulating T-cell activity, phototherapy can further deplete T-cells in psoriatic lesions. Phototherapy has also been shown to decrease Langerhans cells (LC) number, morphology, and function, and this exposure of LC results in suppression of immune response. Such removal of abnormal T-cells may account for, at least in some degree, the generally longer remission period of phototherapy compared to biologics.

In some embodiments, UV phototherapy is administered after discontinuing administration of biologics but before the psoriasis becomes severe. For example, in embodiments in which the psoriasis is reduced from moderate or severe to mild by the administration of biologics, UV phototherapy may be administered before the condition becomes moderate or severe. In certain such embodiments, administration (e.g., routine administration) of UV phototherapy can substantially prevent or delay the condition from relapsing or rebounding to moderate or severe such that the treatment protocol thereafter does not include administration of biologics. For another example, in embodiments in which the psoriasis is reduced from severe to moderate by the administration of biologics, UV phototherapy may be administered before the condition becomes severe. In certain such embodiments, administration (e.g., routine administration) of UV phototherapy can substantially prevent or retard the condition from relapsing or rebounding to severe such that the treatment protocol thereafter does not include administration of the biologics.

The duration between the discontinuing administration of biologics and ensuing phototherapy may vary widely between patients. In certain embodiments, the phototherapy may begin immediately after the discontinuation of the biologic treatment (e.g., immediately after the final application of the biologics). In some embodiments, the phototherapy may begin at least one or more hours, one or more days, or one or more weeks after discontinuing administration of the biologics. In certain embodiments, the phototherapy may begin after discontinuing administration of the biologics but while some biologics remain in the system. In certain embodiments, the phototherapy may begin after discontinuing administration of the biologics and after the biologics have been flushed from the system. Depending on the disease progress after stopping the biologic, the interval between the last biologic treatment and ensuing phototherapy can be less than about two months or greater than about two or more months or even greater than about one or more years. In certain preferred embodiments, the phototherapy begins relatively soon after the discontinuation of the biologics (e.g., immediately or within hours, days, or weeks after discontinuing administration of the biologics) for example to substantially prevent the relapse or rebound of the condition.

Phototherapy has been considered an effective treatment for psoriasis and other skin conditions. However, some unwanted side effects such as cancer and erythema (i.e., sunburn) may occur due to, for example, exposure to the intensity of the light. UV phototherapy in some embodiments utilizes light in the UV-B band, which extends in wavelength between about 280 nanometers and about 320 nanometers. Psoriasis afflicted tissue can be effectively rehabilitated with light having wavelengths between about 300 nanometers and about 310 nanometers. Light having a wavelength spectrum between about 295 nanometers and about 325 nanometers can be effective in healing the tissue as well, although certain wavelengths within that range are more effective than other wavelengths within that range. Therefore, to treat the diseased skin and joints of a subject under conditions that can maximize a likelihood of healing of diseased tissue yet that can reduce or minimize risk of erythema and DNA damage (e.g., cancer), light ranging, for example, between about 295 nanometers and about 320 nanometers, more specifically, between about 300 nanometers and about 310 nanometers, and even more specifically centered around about 308 nanometers may be preferred. In some embodiments, phototherapy doses as high as a subject can tolerate are preferred. More particularly, dosages at least as high as 1 minimum erythema dose (MED) have proven to be extremely advantageous. As defined herein, a minimum erythema dose or MED corresponds to the minimal dosage at which a noticeable change in the normal skin color occurs with distinct edges. The amount of energy necessary to induce redness varies from subject to subject and depends on many factors including race, age, and skin color. Consequently, in treating a particular subject, a level of localized exposure equivalent to 1 MED can be determined for the subject. This level of exposure may be characterized by fluence or the amount of energy delivered to a defined area in, e.g., milliJoules per square centimeter (mJ/cm²) or Joules per square centimeter (J/cm²). Diseased tissue is thereby exposed to doses at least as high as that dose that creates a change in color bounded by distinct edges on healthy skin. Exposure of 1 MED or higher, i.e., doses of about 2 to about 4 or even to about 6 or 8 MED are effective in remedying the diseased condition.

Although the amount of energy that corresponds to 1 MED depends on the skin characteristics of the specific subject, for effective treatment of skin disorders like psoriasis, the recommended fluence of light having wavelengths distributed between about 300 and about 310 nanometers has been determined to range between about 10 mJ/cm² to about 5.0 J/cm². More specifically, the fluences preferably range between about 100 mJ/cm² to about 6 J/cm², and more preferably between about 1 J/cm² and 6 J/cm² (e.g., between about 1.5 J/cm² and 2.5 J/cm² (e.g., about 2.1 J/cm²), between about 4 J/cm² and 5 J/cm² (e.g., about 4.5 J/cm²), between about 4 J/cm² and 6 J/cm² (e.g., about 5 J/cm²). Accordingly, doses as high as about 500 mJ/cm², about 1 J/cm², about 1.5 J/cm², about 2 J/cm², about 2.1 J/cm², about 2.5 J/cm², about 3 J/cm², about 3.5 J/cm², about 4 J/cm², about 4.5 J/cm², about 5 J/cm², about 5.5 J/cm², about 6 J/cm², and values therebetween.

In certain embodiments, phototherapy comprises an average power of between about 2 watts and about 3 watts (e.g., about 2.3 watts), between about 2.5 watts and about 3.5 watts (e.g., about 3 watts), or between about 6.5 watts and about 7.5 watts (e.g., about 7.2 watts). Other average powers are also possible. In certain embodiments, phototherapy comprises an energy between about 8 mJ/pulse and about 15 mJ/pulse or between about 12 mJ/pulse and about 18 mJ/pulse. Other energies are also possible. In certain embodiments, phototherapy comprises a repetition rate of between about 125 pulses/second (Hz) and about 175 Hz (e.g., about 154 Hz), between about 150 Hz and about 250 Hz (e.g., about 200 Hz), or between about 350 Hz and about 450 Hz (e.g., about 400 Hz). Other repetition rates are also possible, and it will be appreciated that higher repetition rates may reduce the treatment duration, for example because the phototherapy device is able to deliver the energy in a shorter period of time. In certain embodiments, phototherapy comprises a treatment area between about 3.5 cm² and about 4.5 cm² (e.g., about 4 cm²). Other areas are also possible. In certain embodiments, phototherapy comprises a dosage of about 600 mJ/cm². In certain such embodiments, phototherapy comprises treating an area of about 4,000 cm² or about 20% of the surface area of the body in less than about 6 minutes (e.g., about 5.5 minutes), treating an area of about 2,000 cm² or about 10% or of the surface area of the body in less than about 3 minutes (e.g., about 3.2 minutes), treating an area of about 1,000 cm² or about 5% or of the surface area of the body in less than about 1.5 minutes, or treating an area of about 500 cm² or about 3% or of the surface area of the body in less than one minute. Other treatment durations are also possible. For example, the duration for higher dosages may be longer and the duration for lower dosages may be shorter. It will be appreciated that the values and ranges of certain variables may change based on the values or ranges of other variables, as well as patient condition, etc. Times reduced to less than about 10 minutes may advantageously help with feasibility of treatment and patient compliance.

Fewer doses of shorter wavelength light are used in comparison to longer wavelength of light. For example, fluences in a range of about 50 mJ/cm² to about 1 J/cm² of light with a center wavelength of about 305 nanometers (e.g., between 304.5 nanometers and 305.5 nanometers) may produce similar results as fluences ranging from about 300 mJ/cm² to about 4 J/cm² of light having a wavelength centered about 310 nanometers (e.g., between 309.5 nanometers and 310.5 nanometers).

As a preferred illustration, the determination of UV dosage using MED is provided in the foregoing, however, this disclosure is not limited thereto. For example, skin pigmentation and the sensitivity of individual subject can be evaluated by other measurements such as Fitzpatrick classifications, slope of the erythemal dose response curve (sED), baseline pigmentation, and tanning response. Fitzpatrick classifications, for example, can score the individual into six varieties (I to VI) of skin types, ranging from extremely pale skinned people who are highly susceptible to burns, to extremely dark-skinned people who may suffer serious discoloration from laser or other light treatment, or other pigment altering therapies or conditions. Those with a higher level of skin type such as V-VI are usually prone to an overactive production of melanin following laser skin or hair treatments. This can lead to permanent discoloration or scarring of the skin, and therefore, people with V-VI type skin may undertake any laser treatment with extreme caution. On the other end of the scale, people with skin type I-II are usually pale and likely to experience severe sun damage from ultra-violet exposure. These skin types are believed to be highly susceptible to skin cancer, and patients are advised to take extreme care to use sunscreen and protect from harmful UV rays. In some embodiments, the subject may be tested for his or her sensitivity to UV and proper dosage for the therapy by any of the foregoing evaluation methods. In some embodiments, more than one evaluation method may be employed to determine optimal dosage of UV, which can enhance or maximize the treatment effect but which can also reduce or minimize the unwanted sunburn and cancer conditions.

In some embodiments, exposing the psoriasis area to high doses of light may treat the condition better than lower dosage light. The number of treatments may be lower with high dose treatments than low dose treatments. Such reduction in number of treatments may thereby decrease in the total quantity of UV-B light to which skin is exposed. The risk of cancer and skin damage depends on the total number of photons or amount of UV-B radiation directed on the skin. Thus, raising the dosage in a single treatment, a couple treatments, or a few treatments can reduce the number of treatments and result in a lower overall number of absorbed photons or UV-B radiation, thereby reducing the risk of cancer and other skin damage. Additionally, lower numbers of treatments may also provide a higher degree of compliance of a subject to an otherwise difficult regimen involving a significant number of visits to the physician. Such a simplification in treatment is favorable, as subjects are less willing or able to adhere to a regimen involving multiple treatments per week for months, years, or a lifetime. As described above, the high dose treatment of UV may be preferred at least in some embodiment, especially when the number of treatments is reduced, the lower dose treatment may be implemented in some other embodiments. For example, a direct correlation has been also observed between occurrence of blisters and particularly successful treatments. For example, levels of exposure to UV radiation as high as 16 to 20 MED, cause UV radiation burns that produce blisters on the skin. However, only a single treatment at this exposure level can rehabilitate the diseased tissue. Thus, employing dosages that cause UV radiation burns accompanied by blistering appears to yield successful single treatment phototherapy.

Since high doses of ultraviolet light enhance the risk of skin cancer and erythema as well as cause other skin damage generally associated with premature aging, the extent of a subject's epidermis to which light is directed may be limited. Light is preferably not delivered to regions of skin other than affected areas, which, particularly with psoriasis, are more tolerant than healthy tissue to higher doses of light within the preferred wavelength regions. In treating psoriasis, for example, UV light is preferably directed onto the lesional as well as surrounding paralesional tissue, which although appearing normal is diseased tissue. Treatment, however, should be restricted only to these affected areas of skin, and areas uninvolved are preferably not exposed to the ultraviolet light. The entire body of the patient is generally not to be subjected to the ultraviolet light, especially when high doses of UV light are employed. In certain such embodiments, the ultraviolet light is delivered to each separate affected region of the body, for example by a handpiece in communication with an excimer laser, as described in U.S. Pat. Nos. 7,276,059 and 7,144,248, each herein incorporated by reference in its entirety. In certain such embodiments, the ultraviolet light is delivered to each separate affected region of the body, for example by a handpiece in communication with one or more UV lamps (e.g., an excimer lamp, an intense pulsed light (IPL) device, an light emitting diode (LED) device). By avoiding treatment of unaffected portions of skin, the dosage can be raised well above conventional dosages as the affected areas will tolerate substantially higher doses without increased risk of side effects. In some embodiments, the high doses of UV illumination are directed to an area on the skin that is between about 25 cm² and about 6,000 cm², between about 25 cm² and about 5,000 cm², between about 25 cm² and about 4,000 cm², between about 25 cm² and about 3,000 cm², between about 25 cm² and about 2,000 cm², between about 25 cm² and about 1,800 cm², between about 25 cm² and about 1,600 cm², between about 25 cm² and about 1,400 cm², between about 25 cm² and about 1,200 cm², between about 25 cm² and about 1,000 cm², between about 25 cm² and about 800 cm², between about 25 cm² and about 600 cm², between about 25 cm² and about 500 cm², between about 25 cm² and about 400 cm², between about 25 cm² and about 300 cm², between about 25 cm² and about 200 cm², between about 25 cm² and about 100 cm², between about 25 cm² and about 50 cm², or ranges included therein. In some embodiments, the high doses of UV illumination are directed to an area on the skin that is less than about 6,000 cm², less than about 5,000 cm², less than about 4,000 cm², less than about 3,000 cm², less than about 2,000 cm², less than about 1,800 cm², less than about 1,600 cm², less than about 1,400 cm², less than about 1,200 cm², less than about 1,000 cm², less than about 800 cm², less than about 600 cm², less than about 500 cm², less than about 400 cm², less than about 300 cm², less than about 200 cm², less than about 100 cm², less than about 50 cm², less than about 25 cm², or values therebetween.

The temporal extent over which exposure occurs can also be important. Exposure of the affected area to the UV light results in heating of the tissue. Unless this heat is sufficiently dissipated, thermal blistering will result. In some embodiments, the high dose of energy provided by the UV light is distributed over a certain length of time. This duration of exposure may, for example, range between about 100 milliseconds and about 1,500 milliseconds for radiation delivered at about 308 nanometers. The illumination is preferably within a short enough time to be practical, yet is long enough to reduce, mitigate, or prevent blistering caused by thermal overload.

In some embodiments, an excimer laser can be employed to generate short high power pulses of light having a wavelength of about 308 nanometers. These pulses can be high in power, e.g., about half a million watts, but short in duration, for example, lasting much less than about 100 nanoseconds (e.g., about 30 nanoseconds). The laser, however, may produce a plurality of such pulses at a repetition rate of about 100, 150, 200, 250, 300, 400, 450, or 500 Hz. Tissue exposed to a plurality of these short pulses will increase in temperature slightly with application of each pulse. The cumulative effect of the plurality of pulses to raise the temperature of the tissue to a certain amount that depends in part on the heat capacity of the tissue. Preferably, therefore, the energy from the laser is spread over a long enough period of time so as to permit sufficient dissipation to avoid excessive build-up of heat from the plurality of short pulses. Thermal damage caused by raising the temperature of the skin above, for example, the blister temperature of 50° C., can thereby be reduced, mitigated, or prevented. The duration of exposure of the affected tissue to the therapeutic doses of UV light, however, depends on the particular dose level. In some embodiments, UV lamps (including UV excimer lamps like the VTRAC® excimer lamp system manufactured by PhotoMedex, Inc. of Montgomeryville, Pa.), intense pulsed light (“IPL”) devices or light-emitting diode (“LED”) devices can be employed to generate the UV light.

The phototherapy may be administered multiple times (i.e., in multiple sessions). In each session, more than one exposure or pulse may be applied to the subject, although a session with a single exposure is also theoretically possible. In general, the subject may have one or more sessions per hour, day, week, month, or year. The number of exposures on each session and frequency of session may change over the course of the treatment at least according to the effect of the therapy. It is likely that the phototherapy at the early stage of the treatment may be more intense and become less intense as the disease condition improves. In some embodiments, the treatment may be applied more frequently (e.g., 2-3 times or sessions per week) at the early stage of the treatment, and may become less frequent (e.g., 2-3 times or sessions per every 2-4 weeks) during the late stage of the treatment. In some embodiments, the dosage may be higher at the early stage of the treatment, and may become lower during the late stage of the treatment.

In some embodiments, UV phototherapy is repeated when needed to keep the psoriasis in a tolerable state of remission. In some embodiments, UV phototherapy continues while psoriasis is in a moderate or mild state. In some embodiments, regularly scheduled UV phototherapy may continue to treat moderate to mild lesions without the patient relapsing or rebounding to severe psoriasis. Once the psoriasis of the subject becomes mild or less, the dosage and/or frequency of the treatment may decrease. In some embodiments, after discontinuing administration of biologics, UV phototherapy may be administered once every 2 weeks, 4 weeks, 2 months, 4 month, 8 months, 12 months, or as often as will keep the psoriasis mild or less.

It is possible that the mild or moderate psoriasis may relapse or rebound while UV phototherapy continues. In some embodiments, the dosage and/or frequency of the UV light may increase to prevent the psoriasis from being more severe again. In some embodiments, administration of the biologics may resume to prevent the psoriasis from relapsing. In certain such embodiments, once the psoriasis is again reduced to moderate or mild by this additional treatment of the biologics, administration of the biologics may again be discontinued and UV phototherapy administered to further reduce the psoriasis and/or to maintain the reduced psoriasis.

In some embodiments, phototherapy is administered without any help from a light-sensitizing agent. In some embodiments, light-sensitizing agents may be used, for example to increase the sensitivity of a cell to UV. In certain such embodiments, one or more light-sensitizing agents may be applied to the subject before or after phototherapy. Examples of light-sensitizing agents include, but are not limited to, coal tar, psoralen, acitretin, and salicylic acid.

In certain embodiments, a device used for administering UV phototherapy may be modified for use in conjunction with or after administration of a biologic. For example, the device may include a software interface or a hardware switch for activating a specific dosage application algorithm to be used after discontinued administration. In certain such embodiments, one or more parameters of the UV light may be modified (e.g., wavelength range, dosage, treatment area, treatment rate, power, combinations thereof, and the like).

In some embodiments, the protocols described herein may be substantially directed by a doctor (e.g., dermatologist) or skin treatment specialist. For example, a doctor may prescribe the biologics for a limited duration, stop prescribing the biologics, and/or prescribe phototherapy thereafter. In some embodiments, the protocols described herein may be substantially directed by a biologics provider (e.g., drug company). For example, a biologics provider may instruct that the biologics should be used for a limited duration, and/or that doctors should no longer prescribe the biologics after a certain treatment level (e.g., as described above). In some embodiments, the biologics provider may suggest phototherapy to treat remedial or lingering conditions. In some embodiments, the protocols described herein may be substantially directed by a phototherapy device provider (e.g., laser company, lamp company). For example, a phototherapy device provider may instruct that biologics should or should not be used for certain conditions (e.g., severe psoriasis), that phototherapy should be used to treat remedial or lingering conditions (e.g., moderate or mild psoriasis), and/or that phototherapy should be used to treat conditions (e.g., moderate or mild psoriasis) after previously conducting some other treatment (e.g., using biologics, reducing from severe psoriasis, reducing psoriasis area by a certain degree). In some embodiments, the protocols described herein may be substantially directed by an insurance company. For example, an insurance company may state that they will only reimburse the care provider for biologics under certain conditions (e.g., as long as psoriasis is severe) and/or that they will reimburse for phototherapy for remedial or lingering conditions (e.g., moderate or mild psoriasis). In certain embodiments, the actors described above may direct these protocols to over about 10 patients, over about 100 patients, over about 1,000 patients, over about 10,000 patients, or more or quantities therebetween.

Certain embodiments described herein are related to serial combinatorial treatment methods comprising administration of biologics and UV phototherapy. Such serial combinatorial methods are expected to provide various benefits to the patient suffering from some of the skin diseases including psoriasis. As described earlier, each of administration of the biologics and UV phototherapy has its own limitations and serious side effects. For example, biologic treatment can be effective in treating large areas, but psoriasis reverts to the pre-treatment state such that patients receive biologics for their entire life. UV phototherapy is an effective local or systemic treatment method of psoriasis; however, it may be less convenient or more difficult to administer to large affected areas. Therefore, relying on the biologics or UV phototherapy alone or even together in parallel may not be an efficient way to treat psoriasis, more particularly, severe psoriasis.

As disclosed herein, administration of these two methods, more particularly, administration of the biologics followed by UV phototherapy, can significantly increase the likelihood of efficient disease control and can also substantially decrease the side effects of individual methods. Biologics are used only to the extent needed to reduce psoriasis until it can be effectively treated with phototherapy. This takes advantage of the ability for biologics to affect large areas, but avoids the side effects and costs associated with continued administration of biologics. Phototherapy is used to further treat psoriasis and/or maintain the condition to the tolerable state of remission. This takes advantage of the ability for phototherapy to efficiently and cost-effectively treat small areas with fewer side effects than biologics.

### EXAMPLES

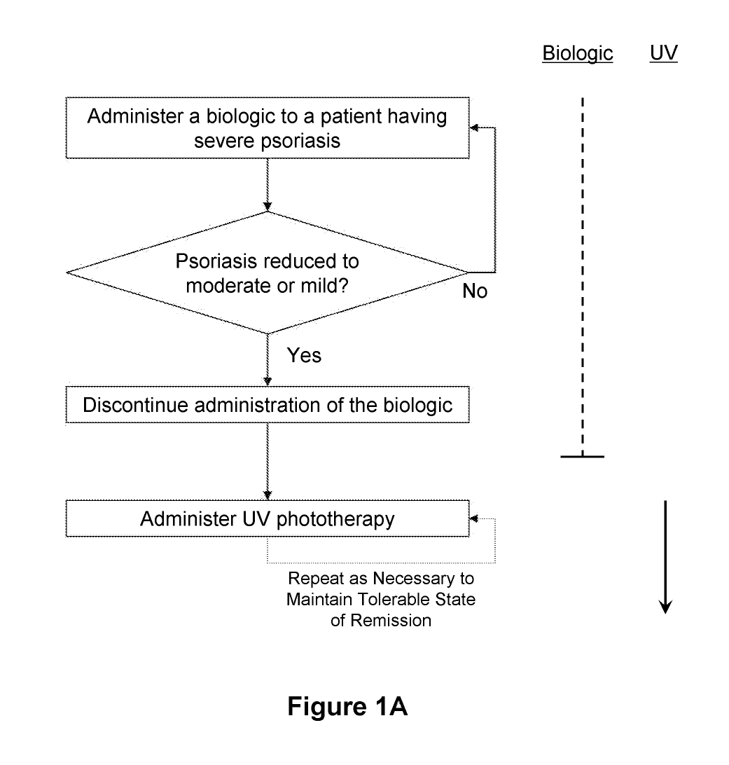

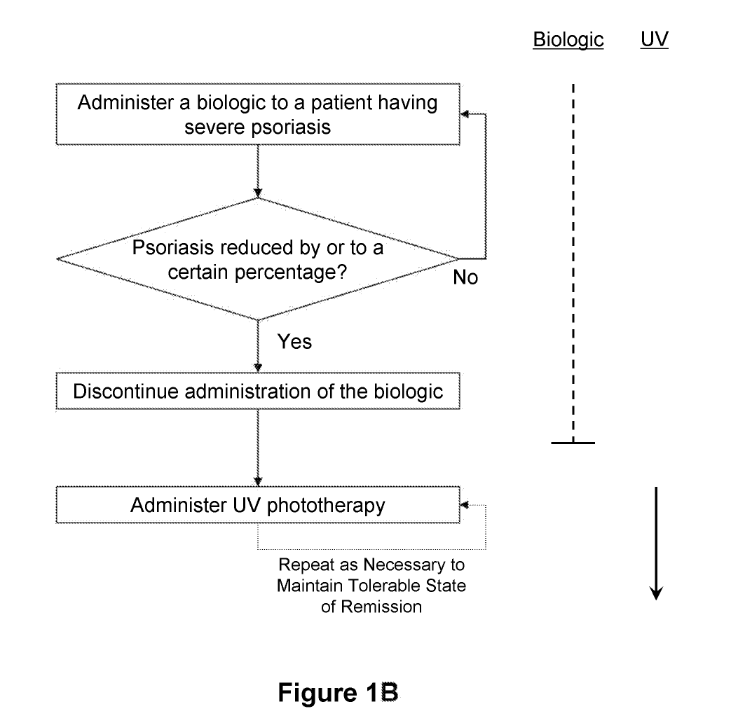

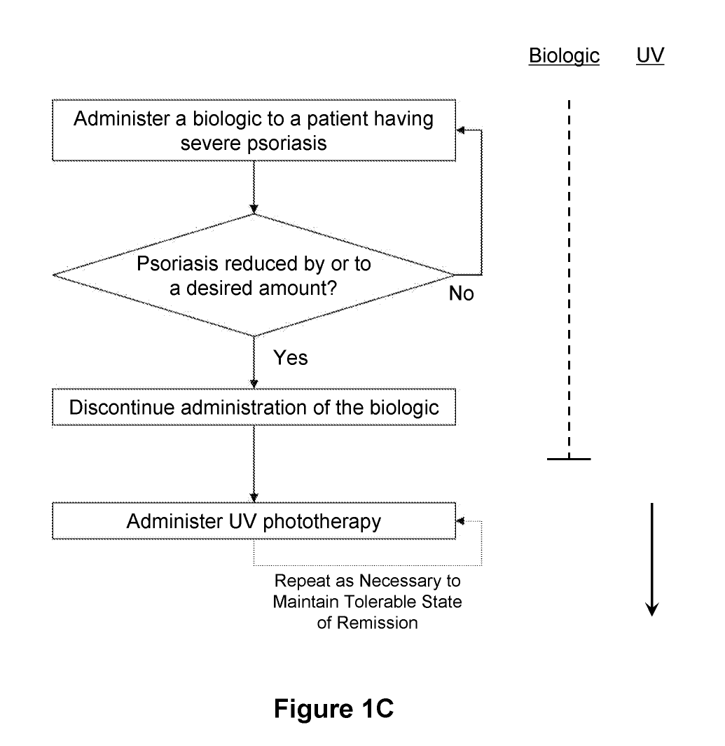

FIGS. 1A-1C

FIGS. 1A, 1B, and 1C illustrate example embodiments of psoriasis treatment protocols. In these embodiments, wherein the patient has severe psoriasis, a biologic may be administered to improve the disease condition. Some details of example biologics, administration pathways, dosages, and treatment frequency are described above. In the embodiment illustrated in FIG. 1A, administration of the biologic may continue at least until the severe psoriasis is reduced to moderate or mild, then is discontinued. In the embodiment illustrated in FIG. 1B, administration of the biologic may continue until the psoriasisis is reduced by or to a certain percentage, then is discontinued. For example, administration of the biologic may continue when the area of the severe psoriasis is reduced by at least about 5%, 10%, 15%, 25%, 30%, 40%, 50%, 60%, 70%, 80%, 90%, 95%, or values therebetween, then is discontinued. In the embodiment illustrated in FIG. 1C, administration of the biologic may continue until the psoriasis is reduced by a desired amount, then is discontinued. For example, administration of the biologic may continue until the psoriasis covers less than about 30%, 25%, 20%, 15%, 10%, 9%, 8%, 7%, 6%, 5%, 4%, 3%, 2%, or 1% of the total body epidermal area of the patient, then is discontinued. In some embodiments, administration of the biologic may continue until the severity of the disease can be maintained, then is discontinued. For example, the biologic may be administered until some symptoms of the psoriasis become stable or reduced, and then the administration becomes discontinued. In some alternative examples, the biologic may be administered until the psoriasis stops spreading or spreads slowly, and then the administration becomes discontinued. The dashed line in each of FIGS. 1A-3C depicts how long the biologic is administered to the patient.

The solid line in each of FIGS. 1A-3C represents a time period of the UV phototherapy, and the arrow end represents that the process may have no predetermined duration or termination. In some embodiments such as those presented in FIGS. 1A, 1B, and 1C, once the psoriasis is reduced and administration of the biologic discontinues, UV phototherapy may be administered. Some details regarding administration of UV therapy are described above. The first UV phototherapy is administered before the psoriasis relapses or rebounds to the severe condition. The UV therapy is configured to further treat the moderate or mild psoriasis. In some embodiments, administration of UV phototherapy begins immediately after administration of the biologic is discontinued. In some embodiments, the solid arrow temporally abuts or at least partially overlaps the dashed line. In some embodiments, administration of UV phototherapy begins at least one or more hours, one or more days, one or more weeks, or more months after administration of the biologic is discontinued (i.e., the temporal space between the dashed line and the solid arrow), but before the psoriasis relapses or rebounds. In some embodiments, administration of UV phototherapy is repeated to treat lesions that may occur. UV phototherapy may be repeatedly administered to the patient at regular intervals or as needed. For example, administration of UV phototherapy may be repeated every month or every several months to ensure the suppression of the disease. For another example, administration of UV phototherapy may be repeated whenever lesions reach a certain size or cause a certain amount of discomfort. The administration of UV phototherapy maintains the disease condition as moderate, mild, or less and prevents the psoriasis from relapsing or rebounding. The UV phototherapy may be the same or different in the repeated administrations. The phototherapy may continue for a relatively long period (e.g., several years or even a lifetime). Other discontinuance criteria are also possible.

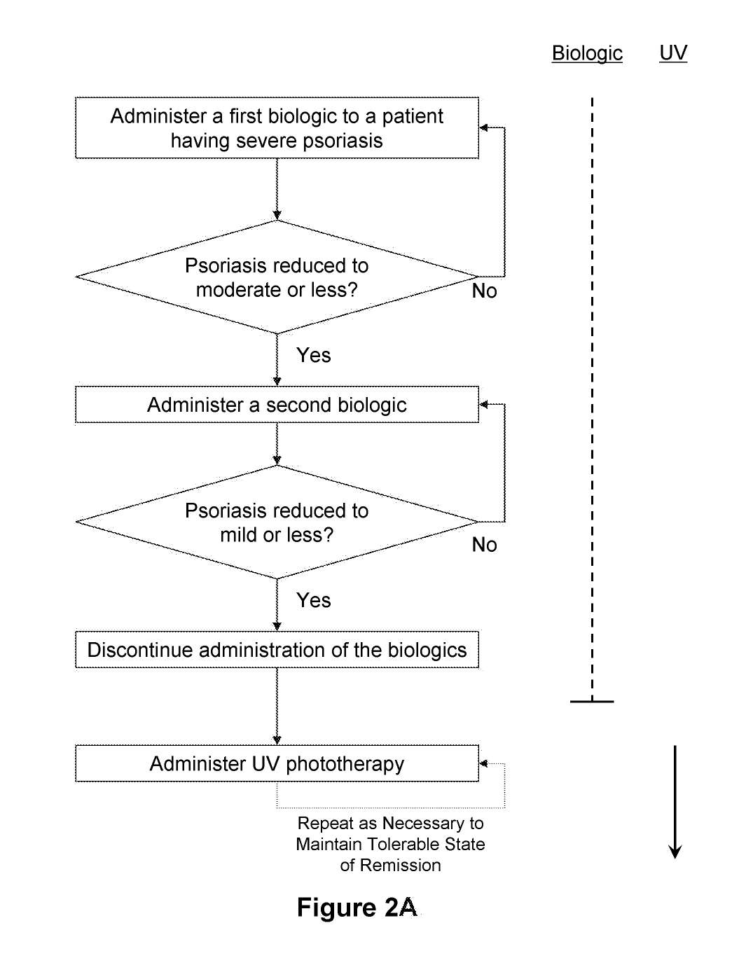

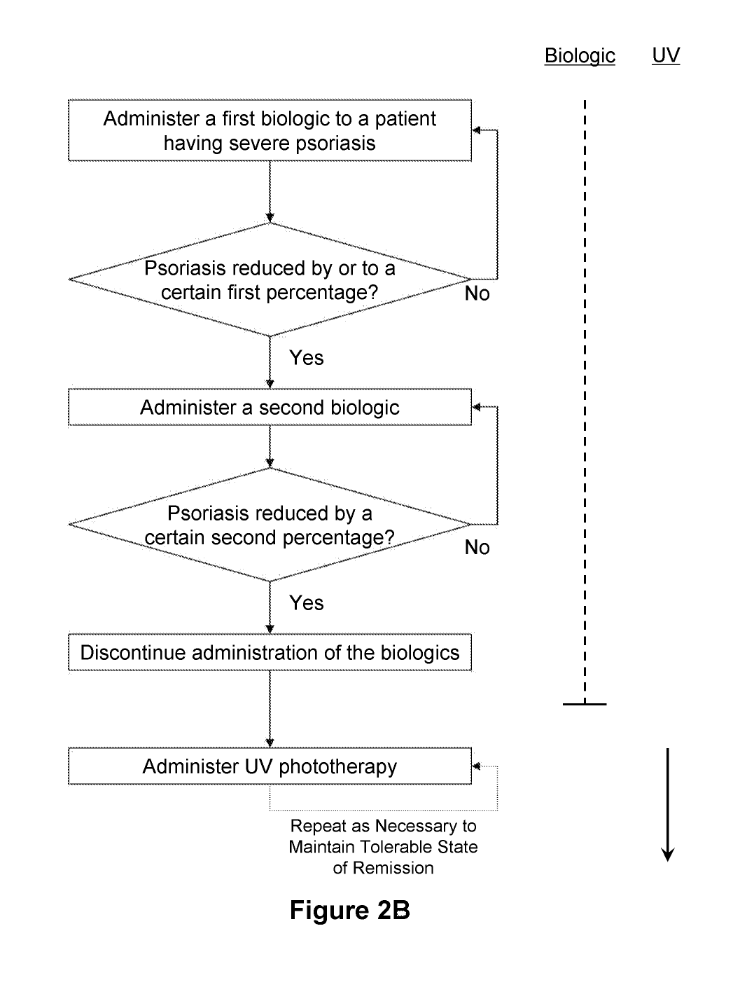

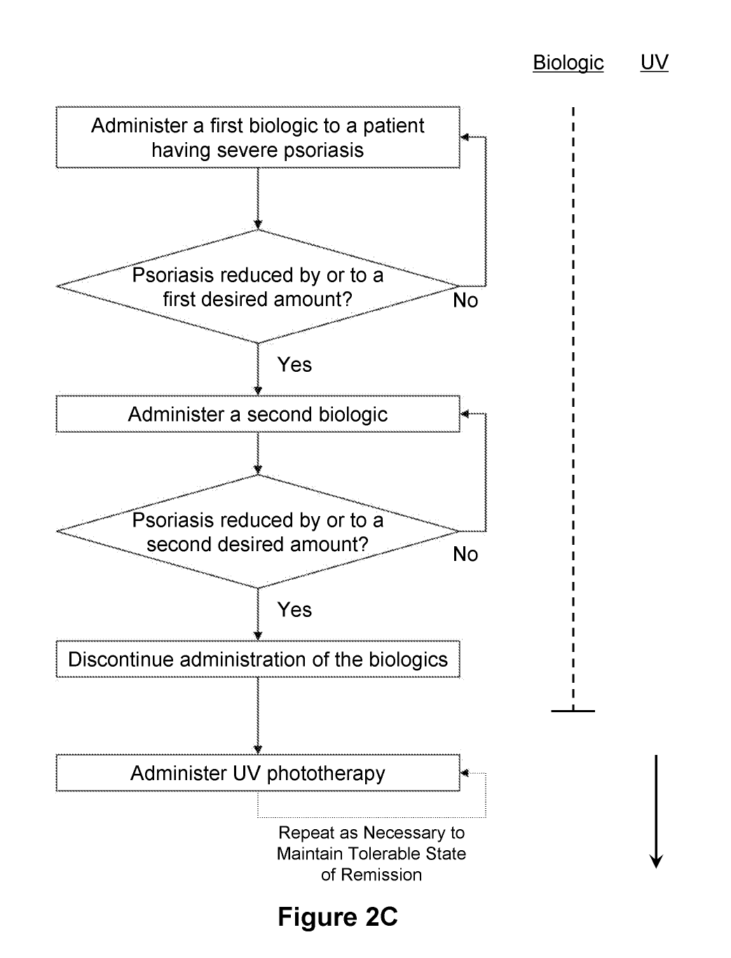

FIGS. 2A-2C

In embodiments depicted in FIGS. 2A, 2B, and 2C, a plurality of cycles of the biologics may be administered to treat the severe psoriasis. A first biologic is administered to suppress the severe disease. The first biologic is discontinued once it reduces the severe psoriasis to moderate or less (e.g., as illustrated in FIG. 2A), by or to a certain first percentage (e.g., as illustrated in FIG. 2B), or by or to a first desired amount (e.g., as illustrated in FIG. 2C). At that point, a second biologic is administered to suppress the moderate disease to be mild or less. In some embodiments, the first biologic is the same as the second biologic. In certain such embodiments, the first biologic has a first dosage and the second biologic has a second dosage different than the first dosage. For example, the second dosage may be about 90% less than the first dosage, about 75% less than the first dosage, about 50% less than the first dosage, or about 25% less than the first dosage. Other dosage differences are also possible. In some embodiments, the first biologic is different than the second biologic (e.g., comprising different active ingredients). The first biologic may be discontinued before the administration of the second biologic (e.g., to prevent interaction) or may be continued with the second biologic (e.g., to enhance effectiveness). Once the psoriasis is then reduced to be mild or less (e.g., as illustrated in FIG. 2A), by or to a certain second percentage (e.g., as illustrated in FIG. 2B), or by or to a second desired amount (e.g., as illustrated in FIG. 2C), administration of all biologics is discontinued. UV phototherapy is repeatedly administered after administration of the biologic is discontinued, for example as described above.

Combinations of these discontinuance criteria are also possible. For example, the first biologic may be discontinued once it reduces the severe psoriasis to moderate or less (e.g., as illustrated in FIG. 2A), and the second biologic may be discontinued once it reduces the psoriasis by or to a certain percentage (e.g., as illustrated in FIG. 2B) or by or to a desired amount (e.g., as illustrated in FIG. 2C). For another example, the first biologic may be discontinued once it reduces the severe psoriasis by or to a certain percentage (e.g., as illustrated in FIG. 2B), and the second biologic may be discontinued once it reduces the psoriasis to moderate or less (e.g., as illustrated in FIG. 2A) or by or to a desired amount (e.g., as illustrated in FIG. 2C). For yet another example, the first biologic may be discontinued once it reduces the severe psoriasis by or to a desired amount (e.g., as illustrated in FIG. 2C), and the second biologic may be discontinued once it reduces the psoriasis to moderate or less (e.g., as illustrated in FIG. 2A) or by or to a certain percentage (e.g., as illustrated in FIG. 2B). Other combinations and discontinuance criteria are also possible.

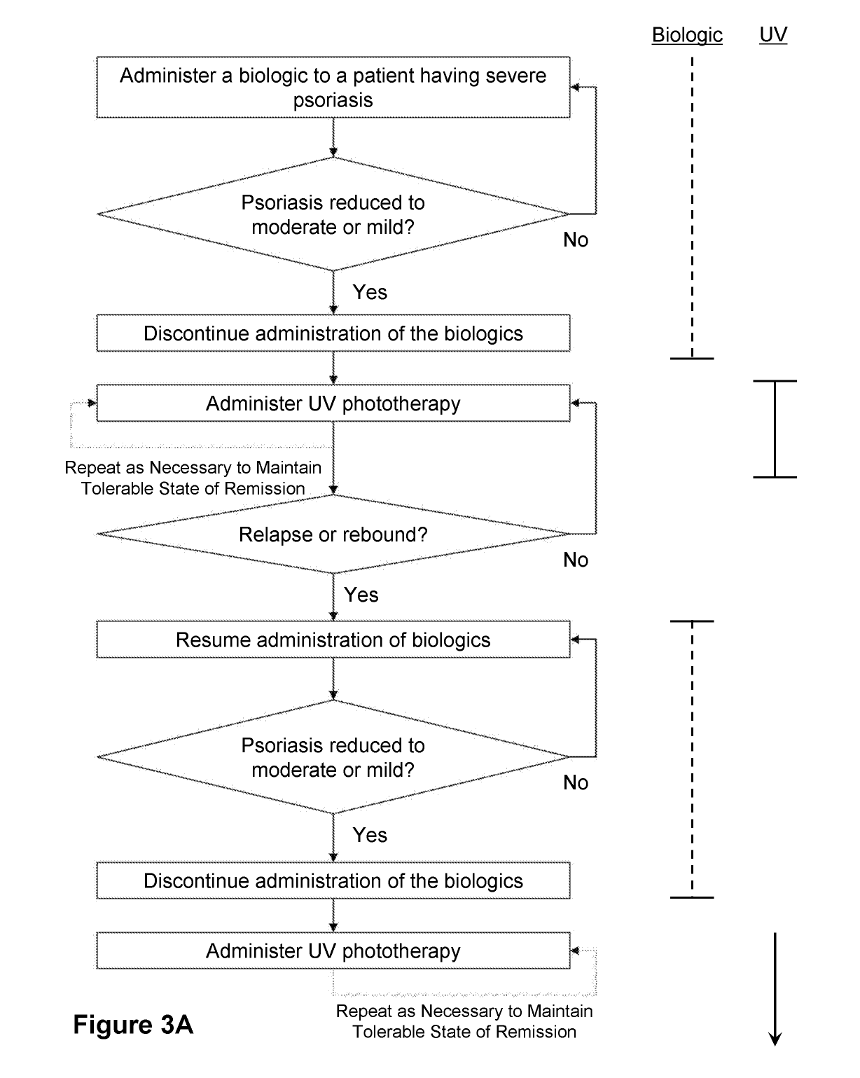

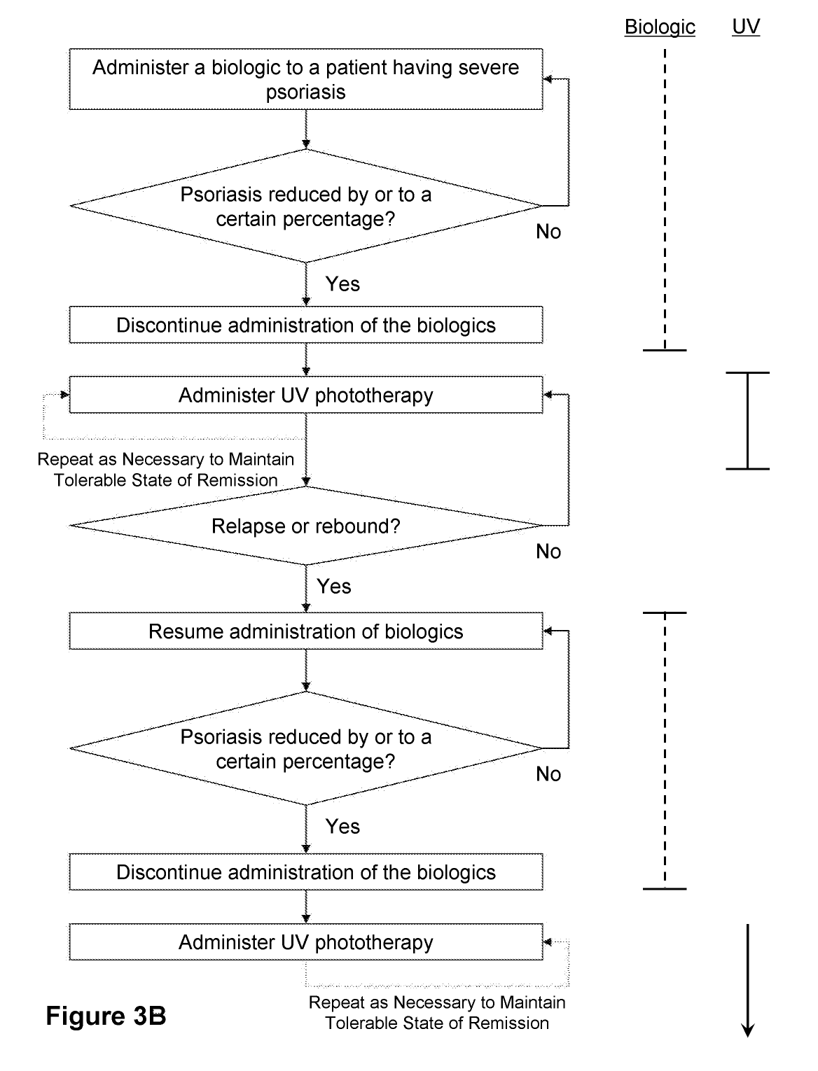

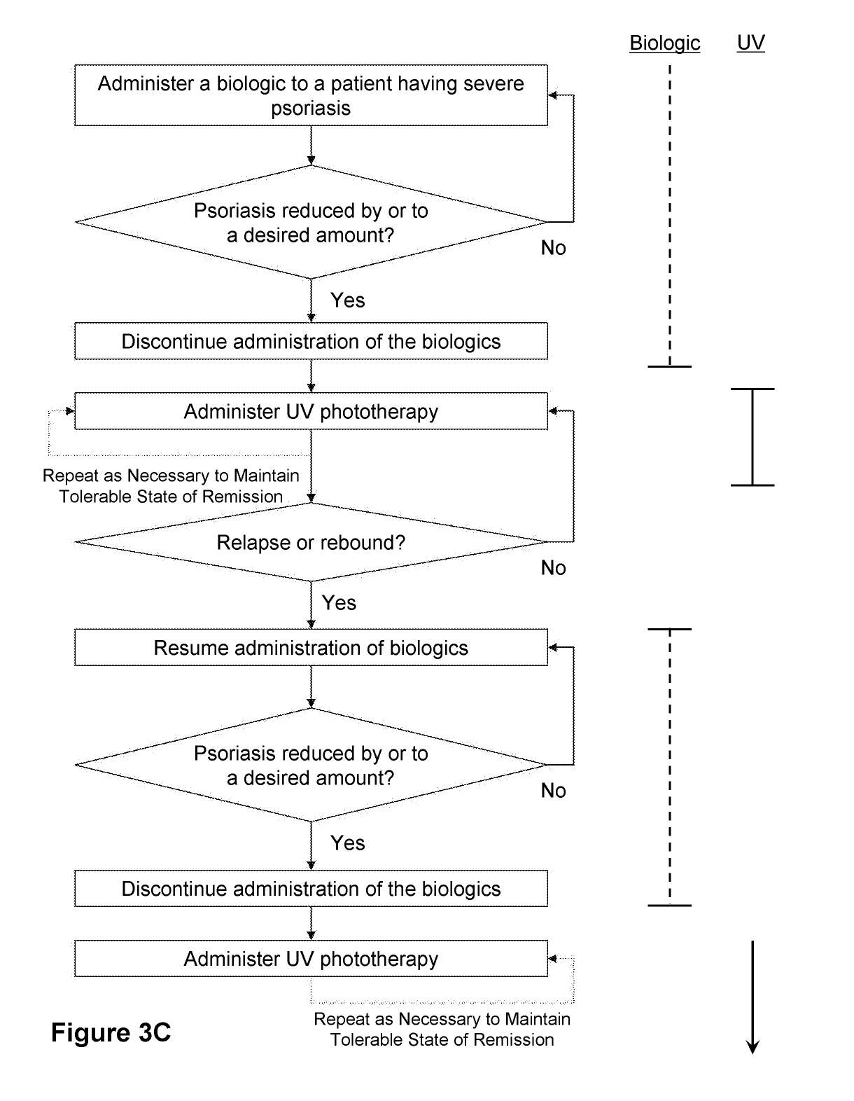

FIGS. 3A-3C

In the embodiments illustrated in FIGS. 3A, 3B, and 3C, there is a chance that the psoriasis may relapse or rebound over the course of treatments, for example for the reasons described above. A biologic is administered to the patient. Administration of the biologic is discontinued once it reduces the severe psoriasis to moderate or less (e.g., as illustrated in FIG. 3A), by or to a certain percentage (e.g., as illustrated in FIG. 3B), or by or to a desired amount (e.g., as illustrated in FIG. 3C). UV phototherapy is repeatedly administered after administration of the biologic is discontinued, for example as described above. If the psoriasis relapses or rebounds to be severe or to some other amount, a biologic may again be administered. The second-round biologics may be the same or substantially the same as the first-round biologics (e.g., due to known effects on the patient), or may be different from the first-round biologics (e.g., because the first-round biologics caused relapse or rebound, even with repeated phototherapy). In some embodiments, the first biologic is the same as the second biologic. In certain such embodiments, the first biologic has a first dosage and the second biologic has a second dosage different than the first dosage. For example, the second dosage may be about 90% less than the first dosage, about 75% less than the first dosage, about 50% less than the first dosage, or about 25% less than the first dosage. Other dosage differences are also possible. In some embodiments, the first biologic is different than the second biologic (e.g., comprising different active ingredients). Administration of the second-round biologic is discontinued once it reduces the severe psoriasis to moderate or less (e.g., as illustrated in FIG. 3A), by or to a certain percentage (e.g., as illustrated in FIG. 3B), or by or to a desired amount (e.g., as illustrated in FIG. 3C). UV phototherapy is again repeatedly administered after administration of the biologic is discontinued, for example as described above. It will be appreciated that checking for relapse or rebound may be incorporated into all of the embodiments described herein, and biologic administration may be repeated for additional rounds if needed. However, biologics should not be continuously administered.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Thus, it is intended that the scope of the invention herein disclosed should not be limited by the particular embodiments described above.

1. A method of treating a subject having psoriasis, the method comprising:
administering UV phototherapy to the subject after discontinuing administering a biologic to the subject after improving severity of the psoriasis.

2. The method of claim 1, wherein the biologic comprises at least one selected from the group consisting of the biologic found in Amevive®, Enbrel®, Humira®, Raptiva®, and Remicade®.

3. The method of claim 1, wherein the biologic comprises at least one selected from the group consisting of alefacept, etanercept, adalimumab, efalizumab, infliximab, and ustekinumab.

4. The method of claim 1, wherein discontinuing the biologic is after the psoriasis is moderate.

5. The method of claim 1, wherein discontinuing the biologic is after the psoriasis is mild.

6. (canceled)

7. (canceled)

8. The method of claim 1, wherein administering the UV phototherapy comprises treating an area of about 2,000 cm².

9. The method of claim 8, wherein administering the UV phototherapy comprises treating the area in less than about 3 minutes.

10. (canceled)

11. (canceled)

12. (canceled)

13. (canceled)

14. (canceled)

15. (canceled)

16. (canceled)

17. The method of claim 1, wherein discontinuing administering the biologic is after the area of psoriasis decreases at least about 50%.

18. (canceled)

19. The method of claim 1, wherein discontinuing the biologic is after a decrease in severity of the psoriasis to covering less than about 30% of the total body epidermal area of the subject.

20. (canceled)

21. (canceled)

22. (canceled)

23. (canceled)

24. The method of claim 1, wherein administering the UV phototherapy comprises directing light propagating from a laser to the patient.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. The method of claim 1, wherein administering the biologic comprises administering two or more biologics.

43. The method of claim 1, wherein administering the biologic comprises a plurality of cycles of the biologic.

44. (canceled)

45. (canceled)

46. (canceled)

47. (canceled)

48. (canceled)

49. (canceled)

50. (canceled)

51. (canceled)

52. (canceled)

53. The method of claim 1, further comprising repeating administering the UV phototherapy at least once per day, at least once per week, at least once per month, or at least once per year.

54. The method of claim 1, wherein parameters for administering the UV phototherapy are at least partially determined based on the skin tone of the subject, Fitzpatrick skin phenotype of the subject, the severity of the psoriasis, the area of exposure, the MED of the subject, or combinations thereof.

55. The method of claim 54, wherein the parameters include at least one of a dosage and a frequency.

56. The method of claim 54, wherein a dosage of the UV phototherapy is equal or greater than about 1 MED.

57. (canceled)

58. (canceled)

59. (canceled)

60. (canceled)

61. (canceled)

62. (canceled)

63. (canceled)

64. (Canceled)

65. (Canceled)

66. (Canceled)

67. (Canceled)

68. (Canceled)

69. (Canceled)

70. (Canceled)

71. (Canceled)

72. (Canceled)

73. (Canceled)

74. The method of claim 1, further comprising continuing administering the UV phototherapy to the subject after the psoriasis becomes mild or in a tolerable state of remission.

75. A method of treating psoriasis, the method comprising:
identifying a patient on a biologic regimen treating psoriasis;
after the biologic regimen reduces severity of the psoriasis, administering a first UV phototherapy to the subject to further treat the psoriasis to be mild or less; and
administering a second UV phototherapy to the subject to maintain the psoriasis to be mild or less.

76. A method of treating a subject having severe psoriasis, the method comprising:
administering a first biologic to the subject;
after improving the severe psoriasis, administering a second biologic to the subject;
discontinuing administering the second biologic after further improving the severe psoriasis; and
after discontinuing administering the second biologic, instructing the subject to undergo UV phototherapy.

77. (canceled)

78. (canceled)

79. (Canceled)

80. (Canceled)

81. (Canceled)

82. (Canceled)

83. (Canceled)

84. (Canceled)

85. The method of claim 76, further comprising, prior to administering the second biologic, discontinuing administering the first biologic.

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (Canceled)

109. (Canceled)

110. (Canceled)

111. (Canceled)

112. (Canceled)

113. (Canceled)
